(19) United States
(12) Patent Application Publication
(10) Pub. No.: US 2011/0091966 A1
(43) Pub. Date: Apr. 21, 2011

(54) Antiviral Peptide Against Hepatitis C Virus

(76) Inventors: Saumitra Das, Bangalore (IN); Renuka Pudi, Rockville, MS (US); Sudhamoni Sonny, Princeton, NJ (US)

(21) Appl. No.: 11/913,311

(22) PCT Filed: Apr. 24, 2006
(86) PCT No.: PCT/IN2006/000141

§ 371 Date: Aug. 26, 2010

(30) Foreign Application Priority Data
May 2, 2005 (IN) 520/CHE/2005

(51) Int. Cl. C07K 14/435; C07K 2/00; C12N 15/12; C12N 15/63

(52) U.S. Cl. 435/320.1; 530/300; 530/325; 536/23.5; 530/324; 530/402

(57) ABSTRACT

Disclosed herein is a small peptide, LaR2C, corresponding to the C terminus of RRM2 of the human La protein that binds to the IRES element of hepatitis C virus RNA and its derivatives. This invention demonstrates that human La protein interacts with the HCV IRES element both in vitro and in vivo and also shown that this interaction enhances the efficiency of viral RNA translation (Pudi et al, J of Biol Chem, 2003). La protein has three putative RNA recognition motifs (RRM1-3). It has been established that RRM2 binds with high affinity around the GCAC sequence near the initiator AUG and the binding induces a conformational change in the HCV IRES which is critical for the internal initiation of translation (Pudi et al, J of Biol Chem, 2004).

This invention relates to a small peptide, LaR2C, corresponding to the C terminus of RRM2 of the human La protein that binds to the IRES element of hepatitis C virus RNA, uses of the peptide in preparing nucleic acid sequence, a polynucleotide, a vector and protein and also novel antiviral agents comprising the said peptide.

### BACKGROUND OF THE INVENTION

Hepatitis C virus (HCV), a member of the Flaviviridae family, is an enveloped positive-sense, single-stranded RNA virus. The 9.6 kb long genome encodes a single polyprotein of about 3,000 amino acids. The polyprotein is processed by host cell and viral proteases into three major structural proteins and several non-structural proteins necessary for viral replication. HCV causes a variety of liver-diseases in humans including liver cirrhosis and hepatocellular carcinoma. It is estimated that about 3% of the world population is infected with HCV and about 85% of infected individuals develop chronic infection.

Translation initiation of HCV occurs in cap-independent manner wherein the ribosomes are recruited onto an internal ribosome entry site (IRES) located mostly within the 5′ untranslated region (UTR) and extending a few nucleotides into the coding region (2). HCV IRES has been shown to form three complex stem-loops and a pseudoknot, which encompasses the initiator AUG codon. Although the HCV IRES binds to the 40 S ribosomal subunit specifically and stably even in absence of any initiation factors, efficient translation requires canonical initiation factors like eIF2 and eIF3 and other non-canonical trans-acting cellular proteins including polypyrimidine-tract binding protein (PTB), La autoantigen, poly (rC) binding protein (PCBP), heterogeneous nuclear ribonucleoprotein L (p68). Recently, binding of a 25 kDa cellular protein (p25) to HCV IRES has been shown to be important for the efficient translation initiation. p25 was originally suggested to be ribosomal protein S9 but later identified as rpS5.

Human La protein is known to interact with HCV IRES and stimulate translation initiation both in vitro and in vivo (3-6). La protein has been shown to specifically interact with both the 5′ and 3′UTR of hepatitis C virus RNA. Sequestration of La in RRL inhibits HCV IRES mediated translation, which can be rescued by exogenous addition of purified La protein. Due to the critical role played by the La protein in HCV IRES translation, the disruption of its interaction with HCV IRES is an attractive target for inhibiting HCV IRES activity. A 60-nucleotide RNA (I-RNA) from the yeast Saccharomyces cerevisiae which preferentially blocked HCV and Poliovirus IRES mediated translation appeared to inhibit the translation by virtue of its ability to bind La protein (4). Recently, it has been shown that a synthetic peptide corresponding to N-terminal ‘La motif’ of human La autoantigen inhibits HCV IRES mediated translation possibly by binding to other essential cellular proteins (5).

### LIMITATIONS OF THE PRIOR ART

HCV establishes persistent liver infection leading to chronic hepatitis, liver cirrhosis and hepatocellular carcinoma. The translation of the positive stranded genomic RNA to produce the viral proteins required for replication is an early obligatory step of the infection process. The translation initiation of the uncapped viral RNA is mediated by the interaction of ribosome and cellular proteins with an internal ribosome entry site (IRES) located within the 5′untranslated region (5′UTR). The translation of the viral RNAs is believed to be controlled by binding of some trans acting cellular proteins to highly structured cis acting RNA elements. Human La autoantigen (originally detected in patients with Lupus Erythematosus) was one of the first IRES trans acting factors (ITAFs) to be identified to interact with Poliovirus IRES element.

Current treatment options in treating HCV involve α-interferon alone or in combination with ribavirin. However, these treatments fail to achieve sustained virological response in majority of patients thus emphasizing the need for novel therapeutic approaches to combat HCV infection (1). Another major limitation is the stability of the peptide inside the cells which cannot be maintained by anything disclosed in the prior art.

### SUMMARY OF THE INVENTION

This invention demonstrates that human La protein interacts with the HCV IRES element in vivo and also shown that this interaction enhances the efficiency of viral RNA translation (6). La protein has three putative RNA recognition motifs (RRM1-3). We have shown that RRM2 binds with high affinity around the GCAC sequence near the initiator AUG and the binding induces a conformational change in the HCV IRES, which is critical for the internal initiation (7).

Since the mechanism ribosome assembly during internal initiation is fundamentally different from the 5′ capdependent translation of cellular mRNAs (8-9), it is exploited as a target for developing antiviral agents. In order to develop effective antiviral agent, we have targeted the La protein interaction with the HCV IRES RNA.

This invention demonstrates a novel approach to inhibit HCV IRES mediated translation using small peptide derived from the C terminus region of RRM2 of La protein. We have shown that a small peptide, LaR2C (24-aminacid long) is capable of binding to IRES element of hepatitis C virus RNA and significantly competes out the interaction of cellular La protein to HCV RNA. The peptide has been shown to prevent the ribosome assembly on HCV IRES and thus effectively block the translation of the viral RNA.

This invention thus provides a small peptide, LaR2C, corresponding to the C terminus of RRM2 of the human La protein binds to the IRES element of hepatitis C virus RNA. The LaR2C peptide competes with the cellular La protein for binding to HCV IRES RNA and act as dominant negative. The LaR2C peptide effectively blocks the ribosome assembly on the HCV RNA. The peptide inhibits the internal initiation of translation of hepatitis C virus in vitro and in vivo. The interaction between the ribosome and HCV RNA could be targeted for designing novel antiviral agents. The sequence of the LaR2C peptide (KYKETDLLILFKDDYFAKKNEERK) or its derivative that blocks ribosome interaction with the HCV-IRES and inhibit viral RNA translation.

This invention also provides a novel antiviral agents comprising the novel peptide LaR2C corresponding to the C terminus of RRM2 of the human La protein that binds to the IRES element of hepatitis C virus RNA and that effectively blocks the ribosome assembly on the HCV RNA and also the use of LaR2C peptide for treatment of any viral infection and particularly Hepatitis C viral infection.

### DETAILED DESCRIPTION OF THE INVENTION:

### Experimental Approach & Results

a. RRM2 of La protein binds to HCV IRES through its C-terminal residues

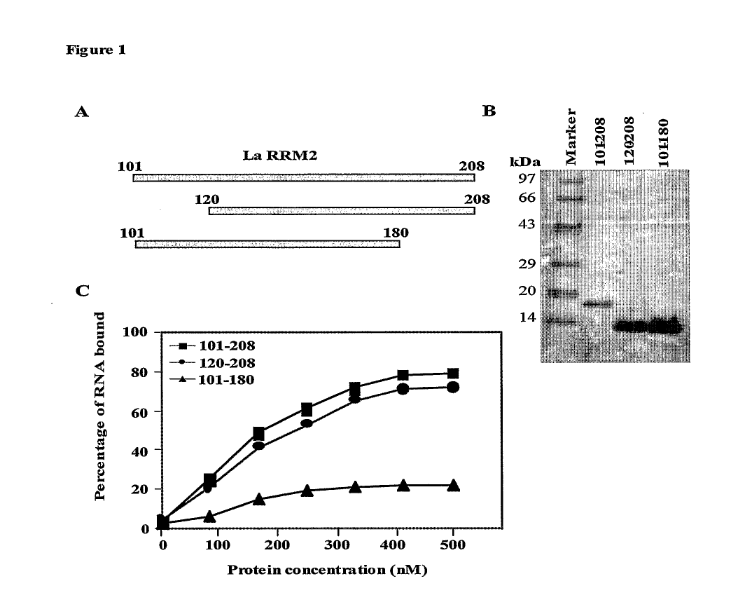

Previously, it has been shown that RRM2 of La protein binds to HCV IRES with high affinity. To precisely identify the region that is important for the binding, two deletion constructs of La-RRM2 (La100-180 and La120-208) with deletions of 20 aminoacids from N-terminus and 28 aminoacids from C-terminal region were generated (FIG. 1A). The over-expressed and purified proteins were analyzed by gel electrophoresis followed by silver staining (FIG. 1B) and used to study their ability to bind HCV IRES RNA using filter-binding assay. [³²P] labeled HCV IRES RNA was incubated with increasing concentration of La-RRM2 (La100-208), La100-180 or La120-208 proteins in RNA-binding buffer. The RNA-protein complexes were bound to nitrocellulose filters and washed with binding buffer to remove unbound RNA. The counts retained were plotted against the protein concentration to obtain saturation curve. The results showed that La120-208 retained the ability to bind HCV IRES RNA with an affinity of 0.16 μM which was comparable to the affinity of La-RRM2 (0.14 μM), while the RNA binding ability was drastically affected in La100-180, where the C-terminal 28 aminoacids were deleted (FIG. 1C). The results indicated that the C-terminal aminoacids of La-RRM2 contribute to the binding with HCV IRES RNA.

b. Peptide LaR2C derived from the C terminus of La-RRM2 is capable of binding to HCV IRES RNA

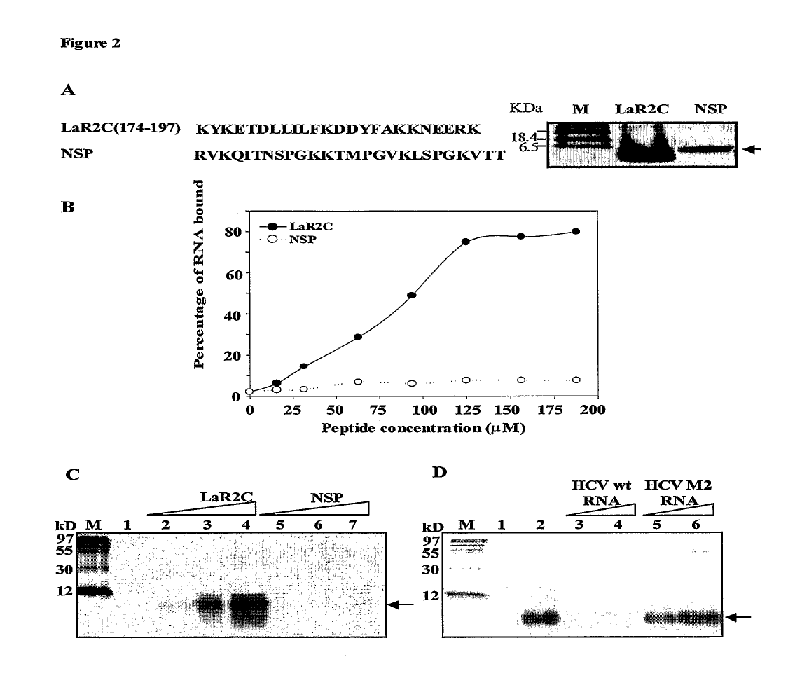

Based on the filter-binding assay results and the information obtained from the computational modeling (6) a synthetic peptide (LaR2C) of 24 amino acids (La174-197), corresponding to C-terminus of La-RRM2, was custom synthesized and tested for its ability to bind to HCV IRES using filter-binding assay. A non-specific peptide of similar length was used as a negative control. The integrity of both the peptides, the La-peptide (LaR2C) and the nonspecific peptide (NSP), were checked by gel electrophoresis followed by silver staining (FIG. 2A). The filter-binding results showed that LaR2C retained the ability to bind to HCV IRES with an affinity of 75 μM, although, the affinity was much lower compared to that of La protein (0.12 μM). The non-specific peptide, NSP, did not show any binding to the RNA (FIG. 2B). The results were further confirmed using UV cross-linking assay where increasing concentration of LaR2C or NSP were used to bind to [³²P] labeled HCV IRES RNA. After the removal of unbound RNA by RNase A treatment, the protein-nucleotidyl complexes were subjected to SDS-15%PAGE followed by phosphor imaging analysis. LaR2C showed efficient binding to HCV IRES RNA in a dosage dependent manner (FIG. 2C, lanes 2-4), while the NSP of similar length did not show any binding (FIG. 2C, lanes 5-7). To study the specificity of the interaction further, competition UV cross-linking assay was performed where the binding of LaR2C with [³²P] HCV IRES was competed with 100 and 200 fold molar excess of unlabeled HCV IRES RNA (self RNA) or with mutant IRES RNA, M2, where GCAC residues in SL IV have been substituted with ACCG (7). The results showed that the unlabeled HCV wild-type IRES RNA efficiently competed out the binding (FIG. 2D, lanes 3-4) confirming the specificity of the interaction of LaR2C with HCV IRES RNA. Even 100-200 fold excess of M2 RNA failed to compete significantly with the [³²P] HCV IRES RNA for binding to LaR2C (FIG. 2D, lanes 5-6) clearly indicating that the LaR2C specifically binds to HCV IRES RNA and perhaps at the same site within the SL IV region where La-RRM2 has been shown to interact.

c. LaR2C binds to HCV IRES in SL IV region similar to La-RRM2

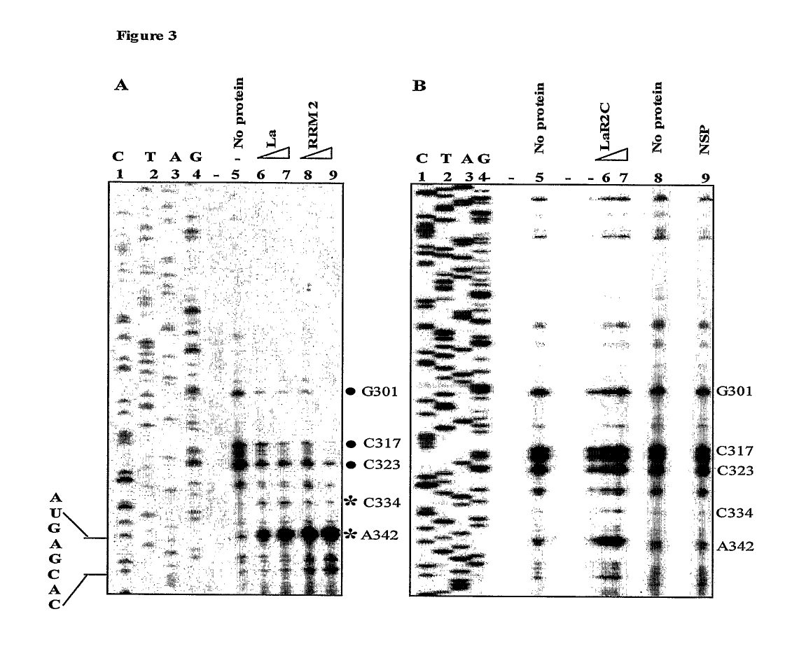

To further confirm the region on HCV IRES RNA where LaR2C binds, primer extension-inhibition assay (toe-printing) was performed using HCV IRES RNA in absence or presence of increasing concentrations of the peptide LaR2C. The full-length La protein and also the RRM2 protein were included in the assay. Increasing concentration of LaR2C or the purified recombinant proteins were incubated with 5 pmoles of in vitro transcribed HCV IRES RNA. To this complex, [³²P] end-labeled primer complimentary to the 3′ end of the HCV-383 was added and extended using AMV-RT. The resulting extended products were analyzed on a 6%polyacrylamide-7M urea denaturing gel. For precise mapping of the contact points, a DNA sequencing reaction corresponding to the HCV 383 RNA and using the same end labeled primer was electrophoresed alongside. The results demonstrated specific RT pauses (toe-prints) with the addition of increasing concentrations of the proteins (FIG. 3) indicating possible binding sites. Two specific toe-prints corresponding to C334 and A342 were observed around the initiator AUG (iAUG) in SL IV region, which showed increase in band intensity with the addition of increasing concentrations of La full-length and RRM2 protein (FIG. 3, panel A).

When LaR2C peptide was added, similar toe-prints (A342 and C334) were obtained around the iAUG within SL IV region (FIG. 3, panel B). However, no significant increases in toe-prints were observed when similar concentration of NSP peptide was used (FIG. 3, panel B). The results indicate that the LaR2C peptide might have contact points within SLIV region at similar positions as observed with the full-length La and RRM2 protein.

d. LaR2C competes with binding of full-length La protein to the HCV IRES RNA

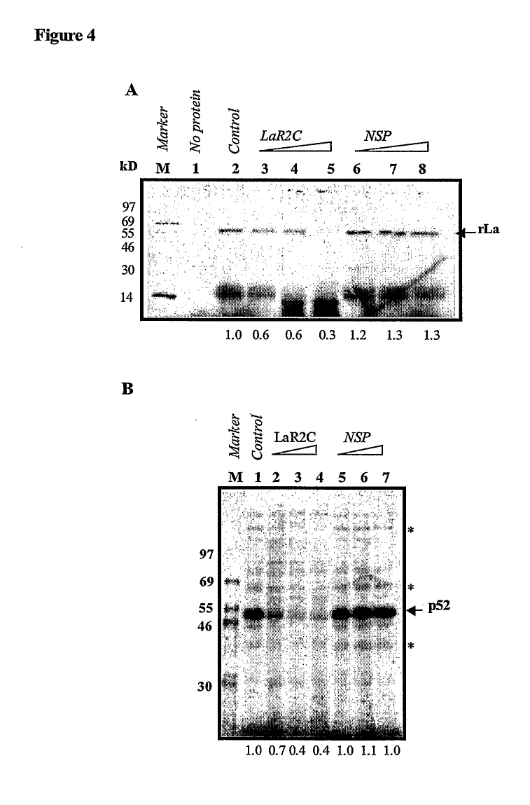

We have shown earlier that RRM2 of human La protein binds to the HCV IRES RNA near initiator AUG (6-7). Since, we have also observed that LaR2C binds to SLIV region of the HCV IRES RNA near the initiator AUG, we were interested to investigate whether LaR2C can compete with the full-length protein for binding to the HCV IRES RNA and if so, what could be the possible consequences. To address this, competition UV cross-linking experiment was performed with purified recombinant La protein and [³²P] HCV IRES RNA in absence or presence of increasing concentration of LaR2C peptide or NSP. The results showed that the binding of La protein to HCV IRES RNA was partially competed out (upto 40%) with the addition 20 μM and 40 μM of LaR2C peptide in the binding reaction (FIG. 4, lanes 3-4). At further high concentration of the peptide (60 μM) the competition was much more pronounced (upto 70%) (FIG. 4A, lane 5). However, the NSP did not show a similar effect (FIG. 4A, lanes 6-8). The result is consistent with the observation that the peptide LaR2C encompassing region of the full-length La protein contributed significantly to its binding to HCV IRES RNA and when added in trans, this region can significantly compete with La protein binding to HCV IRES RNA.

To further verify whether LaR2C peptide can compete with full-length La protein in the context of cytoplasmic extract, UV cross-linking experiment was performed using [³²P]HCV IRES RNA and He La cell extract in absence or presence of increasing concentrations of LaR2C. The results demonstrated that in presence of increasing concentration of LaR2C, the binding of a 52 kDa polypeptide to the [³²P] HCV IRES RNA was found to be drastically reduced (FIG. 4B, lanes 2-4). Immunoprecipitation of the UV cross-linked protein-nucleotide complex using anti-La antibody showed a 52 kDa cross-linked band from He La cell extract, corresponding in size to the 52 kDa protein band observed in the above UV cross-linking experiment (data not shown). Results suggest, the 52 kDa cross-linked band, which showed decreased binding with HCV IRES upon addition of the LaR2C peptide is indeed the La protein. Interestingly, in presence of LaR2C peptide significant changes in band intensities were observed in the binding of some other cellular proteins to HCV IRES RNA (as indicated with asterisks in FIG. 4). The sizes of some of them were found to be similar to that of eIF3 subunits (p170, p66) that were reported to interact with the HCV IRES RNA. From the above observation it appears that the LaR2C peptide can compete with the full-length La protein for binding to the HCV-IRES RNA even in presence of other transacting factors in the He La cytoplasmic extract.

e. LaR2C inhibits HCV IRES mediated translation in vitro

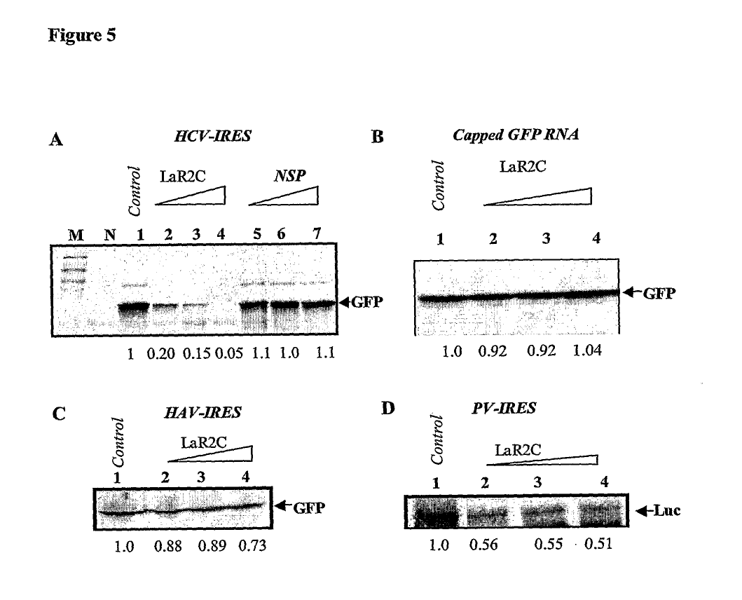

Human La autoantigen has been shown to enhance HCV IRES mediated translation. Since the RNA-protein interaction studies revealed that the peptide LaR2C binds to HCV IRES near iAUG and competes with the binding of full-length La protein to HCV RNA, it was interesting to study whether LaR2C has any effect on HCV IRES mediated translation. For this purpose, monocistronic RNA, HCV-GFP containing the reporter gene GFP downstream of HCV IRES was translated in vitro in rabbit reticulocyte lysate using ^{[35]}S methionine, in absence or presence of increasing concentration of LaR2C (29 μM, 40 μM and 60 μM). The cap-independent initiation of translation, occurring internally from HCV IRES resulted in the synthesis of GFP. The translated GFP was analyzed by electrophoresis followed by phosphorimaging analysis and the band intensities were quantitated by densitometry. It was observed that addition of the peptide LaR2C lead to a significant decrease in the HCV IRES activity (80 to 95%) in a dosage dependent manner leading to reduced synthesis of GFP (FIG. 5A, lanes 2-4). However, addition of similar concentrations of NSP to the translation reactions did not show any significant effect (FIG. 5A, lanes 5-7). Also, the peptide LaR2C didn't affect the translation when capped GFP RNA was used. (FIG. 5B, lanes 2-4), indicating the specificity of the peptide to selectively inhibit HCV IRES mediated translation. Furthermore, addition of similar concentrations of LaR2C peptide didn't show inhibition of HAV IRES (FIG. 5C). However, addition of increasing concentration (20 μM, 40 μM, 60 μM) of LaR2C peptide showed only up to 44-49% inhibition of the PV IRES mediated translation of the reporter gene firefly luciferase. The results suggest that the LaR2C peptide might be more effective in inhibiting HCV IRES compared to other IRES function.

f. LaR2C inhibits HCV IRES mediated translation in vivo

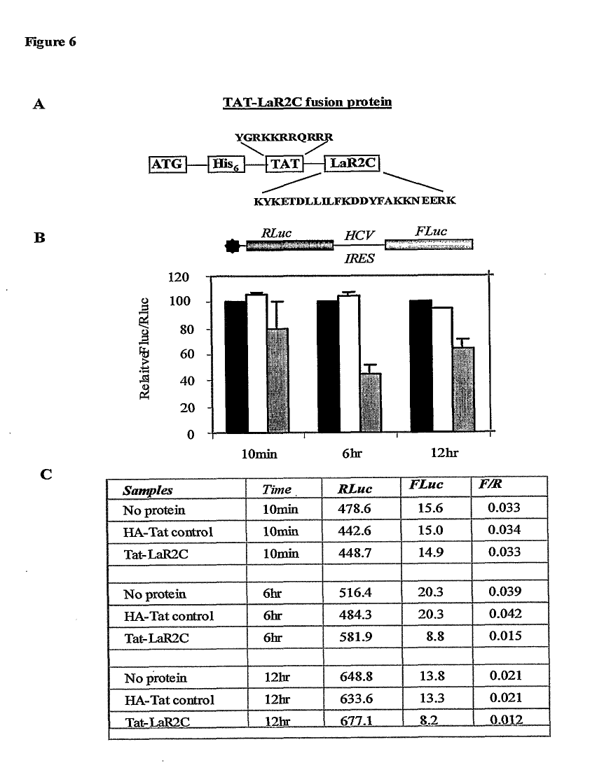

As the LaR2C showed dominant negative effect on HCV IRES mediated translation in vitro, we wanted to investigate whether the same peptide can inhibit the HCV IRES mediated translation in vivo as well. As a first step, sequence coding for the peptide LaR2C was cloned into bacterial expression vector, pTAT which would produce the peptide as a fusion of HIV-TAT peptide that has the property to internalize into mammalian cells when supplied exogenously into the medium (10) (FIG. 6A). Huh7 monolayer cells were first transfected with the pcDNA3-HCV bicistronic construct and the transfectants were selected and maintained in Neomycin containing medium. These selected cells, which constitutively express both the reporter genes, Fluc and Rluc from the HCV bicistronic construct (FIG. 6B), were washed and layered with medium containing 100 nM of TAT-LaR2C fusion protein and incubated for 10 minutes. Similarly, as a negative control, another set of dishes was layered with TAT-HA protein. After incubation the cells were washed and incubated with fresh medium and then harvested at different time intervals, 10 min, 6 hr, 12 hr. The cells were then lysed and Flue and Rluc reporter activities were measured. The relative luciferase activities were represented as a ratio of Flue to Rluc for normalization. The results indicated that, at all the time points, the cells layered with TAT-LaR2C showed a decrease in the HCV IRES mediated translation over the control cells, while the TAT-HA did not have any effect on HCV IRES mediated translation (FIG. 6B). At 6hr post transfection the inhibitory effect was much pronounced (inhibition up to 60%), while the inhibitory effect was found to be gradually decreasing upon prolonged incubation (12 hr time point), possibly due to degradation of TAT-LaR2C within the cells. The results indicated that LaR2C does compete with the interaction of cellular La protein to HCV IRES RNA and exert a dominant negative effect to inhibit HCV IRES mediated translation.

g. LaR2C peptide prevented the assembly of ribosomal complexes on the HCV IRES

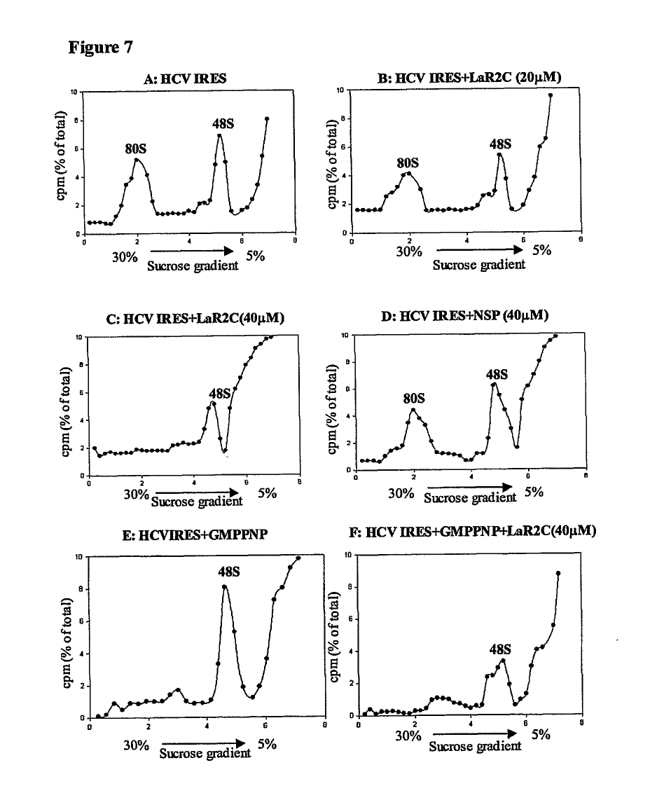

Earlier we have demonstrated that full-length La protein binding at the SLIV triggers a conformational change that facilitates ribosome assembly at the HCV IRES RNA. Since the LaR2C peptide successfully competes with the full-length La protein for binding with the HCV IRES, we have investigated the effect of ribosome assembly upon addition of the LaR2C peptide in the reaction. For this purpose, sucrose gradient centrifugation experiments were performed followed by the analysis of 48 S and 80 S ribosomal peaks. [³²P] labeled HCV IRES RNA was incubated in translation reactions containing RRL and aminoacid mixture and loaded onto 5-30% sucrose gradient and ultracentrifuged for 3 hrs at 30,000 rpm. The fractions were collected from the bottom of the tube and scintillation counts were measured. The percentage of counts in each fraction was plotted against the fraction volume of the gradient. When wildtype HCV IRES RNA was used, two peaks were observed in equilibrium, corresponding to 80 S and 48 S ribosomes associated with the labeled HCV RNA (FIG. 7, panel A). Upon addition of 20 μM of the LaR2C peptide the peaks corresponding to 80 S and 48 S ribosomes were found to be reduced to some extent (FIG. 7, compare panel A with panel B). At higher concentration (40 μM) of LaR2C, the 80 S peak was found to be completely abolished and even the 48 S peak was further reduced compared to control (compare panel C with panel A). However, addition of 40 μM concentration of the non-specific peptide (NSP) didn't show significant alterations in either of the 48 S or 80 S peaks in the ribosomal assembly experiment (FIG. 7, panel D). Interestingly, addition of further high concentration (60 μM) of LaR2C peptide, even the 48 S peak was found to be drastically reduced along with the 80 S peak (data not shown).

It has been demonstrated that in the case of HCV IRES RNA, the 40 S ribosomal subunit can form stable binary complex which is distinct from the 48 S preinitiation complex (8). However, this binary complex might not be separable from the true 48 S peak in the sucrose gradient centrifugation. The actual 48 S prenitiation complex would be competent for the 60 S joining, whereas the binary complex would not lead to functional initiation complex formation. To further clarify, whether the inhibition at the 80 S complex formation by the LaR2C peptide was due to its effect at the preinitiation complex formation, we have performed the ribosome assembly reaction in presence of 10 mM GMP-PNP. GMP-PNP, which is a non-hydrolyzable analog of GTP, inhibits translation initiation at the 48 S stage by preventing the release of eIF2. As expected, addition of GMP-PNP abolished the 80 S peak and caused the accumulation of 48 S ribosome preinitiation complexes in the control reaction (FIG. 7, panel E). Interestingly, when LaR2C peptide (40 μM) was added to the reaction (in presence of 10 mM GMP-PNP), in addition to a drastic decrease in 80 S peak, a significant decrease in the 48 S peak was also observed (FIG. 7, compare panel A, E and F). The results suggest that LaR2C peptide might interfere with the transition step between the HCV-40 S binary complex to the 48 S complex, resulting in formation of defective complex, which is incompetent for 60 S joining. Since addition of LaR2C didn't show accumulation of 48 S peak (as observed in case GMP-PNP), rather it decreases 48 S peak at higher concentration, it seems that the complex might be dissociating eventually.

The result clearly reconfirms the role of La protein in the formation of functional 48 S ribosome complex on HCV IRES, as suggested by our earlier work (ref 6, 7) and also a recent report (11).

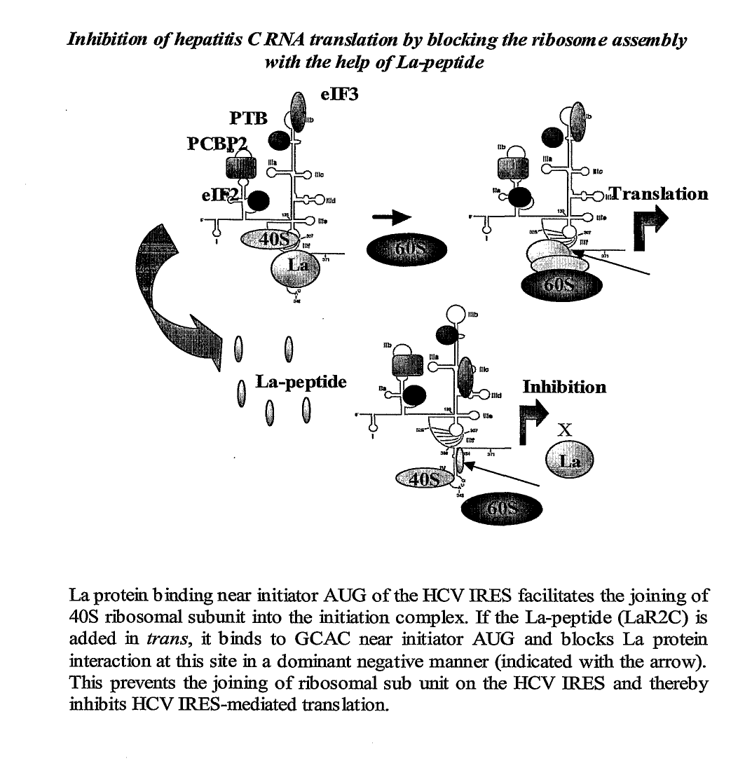

Taken together the results strongly suggest that the LaR2C peptide acts as dominant negative and might compete for the binding of La protein to SL IV region of the HCV-IRES, as a consequence of which the formation of functional ribosomal initiation complex is severely affected resulting in the inhibition of HCV IRES mediated translation (FIG. 8, Inhibition model). From FIG. 8, it can be observed that La protein binding near initiator AUG of the HCV IRES facilitates the joining of 40 S ribosomal subunit into the initiation complex. If the La-peptide (LaR2C) is added in trans, it binds to GCAC near initiator AUG and blocks La protein interaction at this site in a dominant negative manner (indicated with the arrow). This prevents the joining of ribosomal sub unit on the HCV IRES and thereby inhibits HCV IRES-mediated translation.

Figure Legends:

FIG. 1: Effect of deletions on the binding of La-RRM2 to HCV IRES RNA. A: Schematic representation of N- and C terminal deletions within La-RRM2. The corresponding aminoacid numbers of the truncated proteins are indicated. B: The purified protein samples (as indicated on top of the lanes) were analyzed by SDS-10% Tris Tricine gel followed by silver staining. C: Filter-binding assay to study the binding of La 101-208, La 101-180 and La 120-208 to HCV IRES. [³²P] labeled HCV IRES RNA was bound to increasing concentrations of La RRM2 or truncated proteins (as indicated by the symbols within the panel). The amount of bound RNA was determined by binding to the nitrocellulose filters. The percentage of bound RNA was plotted against the protein concentration (nM).

FIG. 2: Ability of the peptide, LaR2C to bind to HCV IRES RNA. A: The sequence of the peptides LaR2C and NSP are indicated. The LaR2C and the NSP peptides were analyzed by resolving in SDS-12% Tris tricine gel electrophoresis followed by silver staining. B: Filter-binding assay to study the binding of the peptide, LaR2C to HCV IRES. [³²P] labeled HCV IRES RNA was bound to increasing concentrations of the peptides, LaR2C or NSP (as indicated by the symbols within the panel). The amount of bound RNA was determined by binding to the nitrocellulose filters. The percentage of bound RNA was plotted against the peptide concentration (μM). C: UV cross-linking of LaR2C and NSP to HCV IRES. [³²P] labeled HCV IRES RNA was UV cross-linked increasing concentration (20, 40 and 60 μM) of either LaR2C or NSP (as indicated above the panel), digested with RNase A and resolved by SDS-15% PAGE followed by phosphorimaging. Lane 1 represents the no protein control. Lane M represents [¹⁴C] protein molecular weight marker. The corresponding molecular masses are indicated to the left of the panel. D: Competition assay to determine specificity of the binding to LaR2C to HCV IRES RNA. LaR2C pre-incubated with 100 and 200 fold excess of unlabeled HCV wild type RNA or HCV M2 RNA (where SL IV region was mutated) as indicated above the lanes, was bound to [³²P] labeled HCV IRES RNA and UV cross-linked. The complexes were treated with RNase A and resolved by SDS-15% PAGE followed by phosphorimaging The band corresponding to LaR2C is indicated to the right of the panels by arrows. Lane 1 represents the no protein control. Lane M represents [¹⁴C] protein molecular weight marker. The corresponding molecular masses are indicated to the left of the panel.

FIG. 3: Primer extension inhibition (toe-printing) analysis. A: HCV IRES RNA (18-383 nt) was incubated with increasing concentrations of La full-length protein (200, 400 ng), La-RRM2 (50-100 ng) or LaR2C (2, 4 μg) as indicated above the lanes, and analyzed by primer extension. Lane 5 shows the no protein control. The toe-prints, which showed increased intensity upon addition of proteins are indicated by asterisks and the toe-prints which showed reduced intensity upon addition of La protein are indicated by filled circles on the right. GCAC motif near AUG is marked on the left of the panel. B: Increasing concentration of the LaR2C peptide (2 and 4 μg, lanes 6-7) or 4 μg NSP (lane 9) were incubated with HCV IRES RNA (18-383 nt) RNA as above and analyzed for the toe-prints. Lanes 5 and 8 represents the no protein control.

The lanes 1-4 in each panel shows the DNA sequencing ladder corresponding to the HCV 18-383 RNA obtained by using the same end-labeled primer. The nucleotides indicated on the right of each panels signifies the corresponding positions on the HCV IRES RNA.

FIG. 4: Effect of LaR2C on the binding of recombinant La protein and other cellular proteins to HCV IRES RNA. A: [³²P] labeled HCV IRES RNA was preincubated with increasing concentrations (20, 40 and 60 μM) of LaR2C or NSP as indicated above the lanes and then bound to recombinant purified La protein. The UV cross-linked complexes were treated with RNase A and resolved by SDS-10% PAGE followed by phosphorimaging Band corresponding to rLa are indicated to the right of the panel. Lane M represents [¹⁴C] protein molecular weight marker. The corresponding molecular masses are indicated to the left of the panel. The intensity of the band corresponding to La protein in each lane was quantitated using densitometry and represented in numbers below the lanes. B: [³²P] labeled HCV IRES RNA was preincubated with increasing concentrations (20, 40, 60 μM) of LaR2C and then bound to HeLa cytoplasmic extract (2.5 μg). The UV cross- linked RNA-protein complexes were treated with RNase A and resolved by SDS-5-15% gradient PAGE followed by phosphorimaging. Bands corresponding to p52 is indicated to the right of the panel. The protein bands whose intensity was reduced are indicated by asterisks. The intensity of the band corresponding to p52 in each lane was quantitated using densitometry and represented in numbers below the respective lanes. Lane M represents [¹⁴C] protein molecular weight marker. The corresponding molecular masses are indicated to the left of the panel.

FIG. 5: Effect of LaR2C on HCV IRES mediated translation in vitro. A: lug of uncapped HCV IRES-GFP RNA was translated in rabbit reticulocyte lysate (RRL) absence (lane 1) or presence of increasing concentrations (20, 40 and 60 μM) of either LaR2C (lane 2-4) or NSP (lane 5-7). The translation of GFP was analyzed on SDS-12.5% polyacrylamide gel followed by phosphorimaging. The band corresponding to GFP is indicated to the right of the panel. B: 1 μg of capped GFP RNA was translated in RRL absence (lane 1) or presence of increasing concentrations (20, 40 and 60 μM) of LaR2C (lane 2-4). The translation of GFP was analyzed on SDS-12.5% polyacrylamide gel followed by phosphorimaging. The band corresponding to GFP is indicated to the right of the panel. Panel C and D: 2 μg of either capped HAV-bicistronic RNA (containing Fluc-HAV-GFP in order) or PV bicistronic RNA (containing Rluc-PV-FLuc in order) was translated in absence (lane 1) and presence of increasing concentrations (20, 40, 60 μM) of LaR2C (lane 2-4). The translation of the reporter genes were analyzed on SDS-12.5% polyacrylamide gel followed by phosphorimaging. The bands corresponding to GFP (representing HAV-IRES function, panel C) and FLuc (representing PV-IRES function, panel D) are only shown in the picture for clarity. The intensity of the GFP band and FLuc band in each lane was quantitated using densitometry and represented in numbers below the respective lanes.

FIG. 6: Effect of TAT-LaR2C fusion protein on HCV IRES mediated translation in vivo. A: Schematic representation of the TAT-LaR2C fusion protein. The aminoacid sequence of TAT and LaR2C are highlighted. B: Huh7 monolayer cells expressing HCV bicistronic RNA were overlaid with 100 nM of either HA-TAT or TAT-LaR2C fusion protein for 10 minutes. The cells were then harvested after different time points (10 min, 6 h and 12 h), lysed and the Rluc and Fluc were measured using Dual Luciferase assay system. The relative ratio of Fluc to Rluc was plotted at each time point. Black bars represent control cells, white bars represent cells overlaid with HA-TAT and gray bars represent cells treated with TAT-LaR2C fusion protein. The HCV bicistronic construct used in the cell line is indicated on top of the panel B. Data from the transfection experiments is expressed as mean ±SD of three independent replicates. C: Absolute levels of RLuc and FLuc activities (in relative light units) of a representative experiment are presented in the table.

FIG. 7: Effect of LaR2C peptide on ribosomal assembly on the HCV IRES RNA. Sucrose gradient sedimentation profiles of [³²P] labeled HCV IRES RNA in absence (panel A) or presence of increasing concentrations of the LaR2C peptide (panel B, and panel C) or the NSP (panel D) after incubation in RRL and separated on 5-30% sucrose gradient. The fractions (200□1) were manually collected from the bottom of the tube and scintillation counts were measured. The counts per minute of each fraction, shown as percentage of the total counts added to the reaction (˜2×10⁵ cpm) and were plotted against the volume of the gradient solution (0-8 ml). The ribosomal peaks corresponding to 48 S and 80 S are indicated. Panel E represents sedimentation profile of HCV IRES RNA incubated in RRL in presence of 10 mM GMP-PNP alone and panel F is that obtained in presence of both LaR2C (40□M) and GMP-PNP (10 mM).

FIG. 8: This illustrates the inhibition of hepatitis C RNA translation by blocking the ribosome assembly with the help of La-peptide.

### ADVANTAGES OF THE PRESENT INVENTION

The translation of the positive stranded genomic RNA to produce the viral proteins required for replication is an early obligatory step of the infection process. The translation initiation of the uncapped HCV RNA takes place through the highly structured IRES element located in the 5′UTR of the viral RNA. Thus the process of IRES-mediated translation is an attractive target for antiviral drug design.

The selective inhibition of HCV IRES-mediated mechanism by LaR2C peptide has a potential to be used as a therapeutic strategy with many associated advantages. Firstly, as the interactions between host cellular proteins and a highly conserved region of the viral RNA is targeted, the chance of generation of viral escape mutants is very low. Approaches like siRNA treatment (RNA silencing) has demonstrated rapid emergence of escape mutants in poliovirus. Although the rate of HCV replication is not as high as that of poliovirus, any sequence-specific antiviral molecule would exert a selection pressure for the generation of escape variants, unlike a strategy targeting host protein-viral RNA interactions. Secondly, the peptide molecule being a part of the host genome, if administered prophylactically to patients harbouring the viral RNA, it is not expected to give rise to non-specific immune responses. Thirdly, as the binding of the cellular proteins is known to be dependent on the secondary structure, more stable derivatives and small molecule structural analogs of the peptide could be utilized. The use of a smaller derivative of the peptide might result in better stability thereby significantly increasing the effectiveness of treatment against Hepatitis C viral infection.

### REFERENCES

1. Choo, Q. -L., G. Kuo, A. J. Weiner, L. R. Overby, D. W. Bradley, and M. Houghton 1989. Isolation of a cDNA clone derived from a blood-borne non-A, non-B viral hepatitis genome. Science. 244: 359-362.
2. Tsukiyama-Kohara, Z., N. Iizuka, M. Kohara, and A. Nomoto. 1992. Internal ribosome entry site within hepatitis C virus RNA. J. Virol. 66: 1476-1483.
3. Ali, N., G. J. M. Pruijn, D. J. Kenan, J. D. Keene, and A. Siddiqui. 2000. Human La Antigen Is Required for the Hepatitis C Virus Internal Ribosome Entry Site-mediated Translation. J. Biol. Chem. 275: 27531-27540.
4. Das, S., M. Ott, A. Yamane, W. Tsai, M. Gromier, F. Lahser, S. Gupta, and A. Dasgupta. 1998. A Small Yeast RNA Blocks Hepatitis C Virus Internal Ribosome Entry Site (HCV IRES)-Mediated Translation and Inhibits Replication of a Chimeric Poliovirus under Translational Control of the HCV IRES Element. J. Virol. 72: 5638-5647.
5. Izumi, R. E., S. Das, B. Barat, S. Raychaudhuri, and A. Dasgupta. 2004. A peptide from autoantigen La blocks poliovirus and hepatitis ,C virus cap-independent translation and reveals a single tyrosine critical for La RNA binding and translation stimulation. J. Virol. 78: 3763-3776.

6. Pudi, R., S. Abhiman, N. Srinivasan, and S. Das. 2003. Hepatitis C Virus Internal Ribosome Entry Site-mediated Translation Is Stimulated by Specific Interaction of Independent Regions of Human La Autoantigen. J. Biol. Chem. 278: 12231-12240.
7. Pudi, R., P. Srinivasan, and S. Das. 2004. La Protein Binding at the GCAC Site Near the Initiator AUG Facilitates the Ribosomal Assembly on the Hepatitis C Virus RNA to Influence Internal Ribosome Entry Site-mediated Translation. J. Biol. Chem. 279: 29879 -29888.
8. Otto G. A., and J. D. Puglisi. 2004. The pathway of HCV IRES-mediated translation initiation. Cell. 119(3): 369-80.
9. Spahn, C. M. T, Kieft, J. S., Grassucci, R. A., Penczek, P. A., Zhou, K., Doudna, J. A and Frank, J. (2001) Hepatitis C virus IRES RNA-induced change in the conformation of the 40S ribosomal subunit. Science, 291, 1959-1962.
10. Becker-Hapak, M., S. S. McAllister, and S. F. Dowdy. 2001. TAT-Mediated protein transduction into Mammalian cells. Methods. 24: 247-56.
11. Costa-Mattioli, M., Y. Svitkin, and N. Sonenberg. 2004. La autoantigen is necessary for optimal function of the poliovirus and hepatitis C virus internal ribosome entry site in vivo and in vitro. Mol. Cell. Biol. 24: 6861-6870.

1. A small peptide, LaR2C, corresponding to the C terminus of RRM2 of the human La protein that binds to the IRES element of hepatitis C virus RNA.

2. The LaR2C peptide that competes with the cellular La protein for binding to HCV IRES RNA and act as dominant negative.

3. The LaR2C peptide that effectively blocks the ribosome assembly on the HCV RNA.

4. The peptide that inhibits the internal initiation of translation of hepatitis C virus in vitro and in vivo.

5. The amino acid sequence of the LaR2C peptide (KYKETDLLILFKDDYFAKKNEERK) or its derivative that blocks ribosome interaction with the HCV-IRES and inhibit viral RNA translation.

6. A polynucleotide comprising the nucleic acid sequence encoding LaR2C peptide (KYKETDLLILFKDDYFAKKNEERK) or its derivative that blocks ribosome interaction with the HCV-IRES and inhibit viral RNA translation.

7. A recombinant vector comprising the polynucleotide as claimed in claim 6.

8. A fusion peptide obtained by cloning of nucleic acid sequence encoding the LaR2C peptide (KYKETDLLILFKDDYFAKKNEERK) and bacterial expression vector, pTAT.

9. A fusion protein TAT-LaR2C comprising the fusion peptide as claimed in claim 8.

10. A novel antiviral agent comprising the novel peptide LaR2C corresponding to the C terminus of RRM2 of the human La protein that binds to the IRES element of hepatitis C virus RNA and that effectively blocks the ribosome assembly on the HCV RNA.

11. The use of LaR2C peptide for treatment of any viral infection and particularly Hepatitis C viral infection.
